(19) United States
(12) Patent Application Publication
(10) Pub. No.: US 2013/0149293 A1
(43) Pub. Date: Jun. 13, 2013

(54) STABLE COMPOSITIONS OF FACTOR IX

(71) Applicants: Jan Jezek, Wellingborough (GB); Barry Kingston Derham, Cambridge (GB)

(72) Inventors: Jan Jezek, Wellingborough (GB); Barry Kingston Derham, Cambridge (GB)

(73) Assignee: ARECOR LIMITED

(21) Appl. No.: 13/591,451

(22) Filed: Aug. 22, 2012

Related U.S. Application Data
(63) Continuation of application No. PCT/GB2011/050365, filed on Feb. 24, 2011.
(60) Provisional application No. 61/307,539, filed on Feb. 24, 2010.

(51) Int. Cl. A61K 38/48

(52) U.S. Cl. CPC A61K 38/4846
USPC 424/94.3

(57) ABSTRACT

The invention allows substantial improvements in stability of coagulation Factor IX in aqueous compositions. An aqueous composition sealed in a non-glass container comprising Factor IX in a buffer and calcium ions is provided, together with methods of stabilizing an aqueous Factor IX composition comprising storing said composition in a non-glass container for at least 7 days.

### FIELD OF THE INVENTION

This invention relates to the stabilization of coagulation Factor IX, particularly in aqueous liquid compositions for therapeutic applications.

### BACKGROUND TO THE INVENTION

Factor IX is a globular protein which has a molecular weight of about 70,000 daltons and which, in a normal individual, is constantly produced in the liver and circulates at a normal blood plasma concentration of approximately 5 μg/ml.

Hemophilia B is a very serious illness which results in decreased in vivo and in vitro clotting activity and requires extensive medical monitoring throughout the life of the affected person. Such persons show normal clotting times only when provided with exogenous Factor IX which is extracted from the blood plasma of normal individuals. Except for such treatment, the afflicted person can suffer from spontaneous bleeds in joints which produce severe pain and debilitating immobility, bleeds into muscles resulting in large volumes of blood accumulating in the tissue, spontaneous bleeds in the throat and neck which may cause asphyxiation if not immediately treated, bleeding into the urine, and severe bleeding following surgery or minor accidental injuries or dental extractions.

Functional Factor IX deficiencies can arise in different ways. The gene coding for Factor IX is located on the X chromosome. This explains why hemophilia B is much more common in males than females. Some of the afflicted persons are known to have inherited an X chromosome with a complete deletion of the Factor IX gene. These severely affected persons may even produce antibodies to therapeutically injected Factor IX. Many hemophilia B patients are known to produce a Factor IX molecule with an altered amino acid sequence which results in molecules of partial or no coagulation activity. Some hemophilia B patients produce normal Factor IX, but in insufficient quantities to effect clotting within a normal time after injury.

As mentioned above, Factor IX activity can be restored in the patient by injection of normal human plasma. However, at minimum, several liters would have to be administered to raise the patient's circulating Factor IX levels to an effective range. Accordingly, the emphasis in therapy for hemophilia B patients has been to provide injections of a plasma concentrate highly enriched in Factor IX or injections of Factor IX preparations prepared by recombinant technology.

Therefore a need exists for stable high concentration Factor IX liquid compositions for injection.

Certain stabilised formulations of metalloproteins containing calcium ions are disclosed in WO2009/133200 (Jezek).

U.S. Pat. No. 5,770,700 (Webb) describes a formulation of Factor IX containing arginine and citrate. It is stated that inclusion of calcium ions at the concentration tested (5 mM) is destabilising and is to be avoided (see the conclusion at the end of Example 1).

U.S. Pat. No. 5,925,738 (Miekka) discloses aqueous formulations of plasma proteins including Factor IX together with a pH buffering compound, calcium ions and an osmotic modulating agent (such as NaCl) in a concentration of 1-500 mM. The most preferred concentration of calcium ions is said to be between 10 mM and 100 mM. One exemplified formulation (Example 12) contains Factor IX (100 clotting units/mL), 10 mM histidine, 0.10 M NaCl and 10 mM calcium chloride at pH 6.2.

### SUMMARY OF THE INVENTION

The invention allows substantial improvements in stability of coagulation Factor IX in aqueous compositions. Factor IX products are currently presented as a lyophilized formulation of Factor IX either produced by recombinant technology or purified from pooled plasma. The administration of the lyophilized product is a very complex procedure involving a number of steps. Administration must be done within 3 hours of reconstitution. Reconstitution must be done carefully to avoid damage to the product. A stable aqueous formulation of Factor IX would allow the development of a convenient patient-ready pre-filled syringe or a pump-delivered formulation, replacing the current formulations. A preferred formulation is stable at room temperature; however, even an aqueous formulation stable at 5±3° C. would be a very important advancement in terms of convenience of administration.

The invention discloses stable aqueous compositions of Factor IX in which the potency of Factor IX is preserved on a prolonged incubation both at 5±3° C. and at 25±2° C.

The invention is based on the discovery that stable Factor IX formulations ensure appropriate binding of calcium cation in the structure of the protein, whilst ensuring that there is no unacceptable level of self-activation of Factor IX during storage. The desired features are summarized as follows:
1. The key components of the formulation improve the stability of proteins which contain metal ions, such as calcium ion, in their three dimensional structure. WO2009/133200A1, to Jezek, describes controlling metal ions in proteins generally. The γ-carboxylic rich region of Factor IX binds calcium ions, which are essential for proper interaction of Factor IX with phospholipid membranes and ultimately for proper function of Factor IX. It has been suggested (Huang et al.: J. Biol. Chem. 279(14), 14338-14346, 2004) that calcium binding results in a more ordered and stable structure of the γ-carboxylic region. It is therefore important to maintain the appropriate binding of the calcium cations within the structure of Factor IX by selecting components in the formulation that cause minimal interference with the binding.
2. The preferred buffering system for Factor IX is a combination of TRIS and benzoate anion, at pH around 6.8. However, other buffering systems can be used. The preferred tonicity modifiers are NaCl and 1,2-propanediol, but a number of other components can be used.
3. Unlike the observations made in U.S. Pat. No. 5,770,700, we have found that the use of calcium cation is advantageous in compositions of Factor IX. Importantly, however, if calcium ion is used, then its concentration must be kept below 1 mM. Higher concentrations of calcium cation lead to unacceptable self-activation of Factor IX, even in a formulation optimized with respect to other components and the container. This is a consideration apparently not considered by Miekka in U.S. Pat. No. 5,925,738. Preferably, the concentration of Ca²⁺ is between 0-1 mM, more preferably between 0.1-0.7 mM, most preferably between 0.2-0.5 mM. Preferably, the calcium cation is accompanied by a strong ligand, such as EDTA at a concentration that does not exceed the concentration of the calcium cation and is ideally about one tenth of that of the calcium cation (e.g. 0.5 mM Ca²⁺+0.05 mM EDTA). The small amount of the strong ligand removes traces of other unchelated metal ions from the composition.
4. The composition of Factor IX must be kept in a non-glass container that is sufficiently robust to withstand sterilization (e.g. by heat or radiation) prior to aseptic filling with the Factor IX formulation. The preferred containers for Factor IX compositions are plastic ones, such as polyethylene or polypropylene containers. However, containers made from a number of other non-glass materials can be used. The use of a glass (e.g., Type I borosilicate glass) container results in unacceptable self-activation of Factor IX, even in the absence of calcium cation. In addition, using plastic containers in combination with high calcium level also leads to increased self-activation (although much less significant than using a glass container with the same level of calcium cation). Consequently, in order to keep the Factor IX stable it is essential to ensure both low calcium level and a non-glass container.

Factor IX self-activates in vivo by interaction with negatively charged phospholipids and calcium ions. Without being bound to theory, it is believed that the negative charge at the surface of glass containers can initiate and/or accelerate self-activation of Factor IX. It is believed that some surface modifications (such as siliconization) may prevent/reduce the self-activation rate caused by glass, so such containers should also be considered for stabilized Factor IX compositions.

Thus according to the invention there is provided an aqueous composition sealed in a non-glass container comprising Factor IX in a buffer and calcium ions at a concentration of less than or equal to 1 mM and wherein the composition is free or substantially free of the free forms of excipients which are medium-strength ligands or strong ligands.

### DETAILED DESCRIPTION OF THE INVENTION

The invention is based on the discovery that control of calcium ions and other formulation components, together with maintaining the formulation in a non-glass container provides stable aqueous formulations of Factor IX. Calcium ion plays an important structural role in the molecule of Factor IX. It is therefore important to maintain the bond between the protein and the calcium ion in order to keep Factor IX in a stable form. Hence presence of calcium ions in the aqueous compositions of Factor IX can be beneficial. It is also critical that other formulation components exhibit minimal interference with the binding of calcium within the structure of Factor IX. This can be achieved by avoiding free forms of excipients that have a strong ability to bind calcium ions (medium strength or strong ligands). Presence of such excipients in a form that is bound to calcium ions present in the bulk aqueous medium (i.e. not calcium ions bound within the structure of Factor IX) is permitted in Factor IX compositions according to the present invention as the ability of such bound forms to interfere with bonds between protein and calcium is limited.

Importantly, the presence of calcium ions can contribute to the undesirable self-activation of factor IX, and it is critical to maintain the concentration of calcium within narrow limits as defined by the present invention.

The invention uses a non-glass container. A non-glass container is intended to include any container where the surface(s) exposed to the aqueous Factor IX composition is not borosilicate glass or other conventional glass material. The container can be selected from plastic materials, such as polypropylene, polyethylene, polypropylene-polyethylene copolymers, polycarbonate, polystyrene or thermoplastic polyester. Alternatively, glass containers with surface modifications eliminating the negative charge (such as siliconized glass) can also be used.

The excipients, such as buffers and tonicity modifiers, selected for the composition are preferably weak ligands. The composition is free or substantially free of free forms of excipients which are medium-strength ligands or strong ligands. Where the composition contains medium strength and strong ligands, their concentration must be lower than the concentration of metal ions (e.g., calcium ions) in the composition and, preferably, are not in free form when present (e.g., they are complexed to excess metal ions).

The term “free form of a ligand” is used herein to describe molecules of a ligand which is not bound to a metal cation in a particular composition comprising ligand molecules and metal ion molecules. One of ordinary skill in the art will be able to calculate the proportion of free ligand from stability constants of the ligand-metal ion complex provided that that overall concentrations of all ligands and all metal ions in the composition are known.

The term “ligand” is used herein to encompass any compound capable of binding metal ions resulting in formation of complex ions. For the purpose of this invention the ligands are divided into “weak ligands”, “medium-strength ligands” and “strong ligands”. The terms of “weak ligand”, “medium-strength ligand” and “strong ligand” are defined based on the stability constants of their complexes with calcium ion, as follows: A weak ligand has a stability constant of a complex with calcium ion log K<0.5; a medium-strength ligand has stability constant of a complex with calcium ion log K between 0.5 to 2; a strong ligand has stability constant of a complex with calcium ion log K>2. All stability constants referred to herein are those measured at 25° C.

The stability constants of metal-ligand complexes can be obtained from a comprehensive database published by the US National Institute of Standards and Technology (NIST Standard Reference Database 46, R. M. Smith and A. E. Martell: Critically Selected Stability Constants of Metal Complexes Database). The art of using the stability constants in the context of the present invention is described in detail in WO2009/133200A1, which is incorporated herein by reference.

Examples of suitable weak ligands (calcium ion log K values are in parentheses) include: benzoate (0.20), salicylate (−0.87), tris(hydroxymethyl)aminomethane (0.25) and chloride (0.1). Another example is imidazole (−0.1). The concentration of the weak ligands in the composition is typically between 0 mM 1M, preferably between 1 mM 0.5 M, more preferably 5-100 mM, most preferably between 5-50 mM.

Preferably, the final composition is approximately isotonic.

Examples of suitable medium strength and strong ligands (calcium ion log K values are in parentheses), when used as described above, include: EDTA (10.81), citrate (3.48), histidine (1.21), lysine (1.05), ornithine (1.68), methionine (2.04), cysteine (2.5), glutamate (1.43), tyrosine (1.48), aspartate (1.7), alanine (1.3), glycine (1.09), glycylglycine (1.24), malate (2.06), phthalate (1.6), maleate (1.76), lactate (1.48), glycolate (1.11), triethanolamine (0.87), carbonate (3.22), borate (1.76) and sulphite (2.62). Another example is acetate (0.55).

The selection of ligands is described generally in WO2009133200A1.

A strong or medium ligand (preferably a strong ligand) can optionally be added to the composition to control or minimize undesirable protein-metal ion complexation. Thus, the preferred amount of ligand to be added is that which binds undesirable metal ions (e.g., residual or trace transition metals, such as copper, zinc or iron) and excess calcium ions. However, the preferred amount of ligand is preferably not so great as to compete with and prevent desirable calcium ion complexation to the Factor IX protein. This preferred range of ligand is defined herein as an “effective amount.” For example, EDTA can be added at a concentration no greater than the total concentration of calcium ion in the composition, and is ideally about one tenth of the total concentration of calcium ion so as to allow a significant portion of the calcium ions to exist in free form. For example, the composition comprises EDTA at a concentration between 0.001 mM to 0.1 mM. A suitable strong ligand that may be added for this purpose is EDTA however more generally the most suitable strong ligands have a calcium ion log K value measured at 25° C. of 5 or more e.g. 8 or more e.g. 10 or more.

The composition optionally contains calcium cations at a concentration of less than or equal to 1 mM, such as 0.1 to 1 mM, preferably between 0.1-0.7 mM, most preferably between 0.2 to 0.7 e.g. 0.2-0.5 mM. Another suitable range is 0.4 to 0.6 mM especially around 0.5 mM. Preferably, the calcium cation is accompanied by a strong ligand, such as EDTA (or such as another strong ligand having a calcium ion log K value of 5 or more e.g. 8 or more e.g. 10 or more) at a concentration that does not exceed the total concentration of the calcium cation and is ideally about one tenth of that of the calcium cation (e.g. 0.5 mM Ca²⁺+0.05 mM strong ligand such as EDTA). As noted above, the small amount of the strong ligand removes traces of other metal ions from the composition.

The stability achieved is measurable by the % changes in residual potency of Factor IX (APTT test) after incubation at targeted storage temperatures for several weeks or up to around six months or more, without any significant self-activation (NAPTT test). For example, the composition shows a residual potency within 10% of a Control Composition after incubation at 25° C. for 20 weeks and a response time of >200 s in the NAPTT test.

In order to make an aqueous composition suitable for therapeutic application, such as intravenous, subcutaneous or intramuscular application, certain desirable characteristics of the composition must be ensured, such as safety and regulatory acceptance of the excipients. The key aqueous compositions of Factor IX disclosed herein are ideally based on excipients already approved by regulatory authorities as inactive ingredients in drug products.

A liquid composition for therapeutic use must be sterile. Sterility of a liquid composition for therapeutic use can be achieved by filtering the composition prior to the final filling to an appropriate container, such as a vial or a pre-filled syringe, under sterile conditions, using an appropriate filter or membrane, such as a 0.22 μm filter or a 0.45 μm filter. The key aqueous compositions of Factor IX disclosed herein are preferably sterile-filtered and filled aseptically into the final container.

The objective of the present invention is an aqueous formulation of Factor IX in which the potency of Factor IX is preserved for extended period of time both at 5±3° C. and at 25±2° C. while there is no unacceptable level of Factor IX activation. The invention is applicable to recombinant Factor IX as well as Factor IX purified from pooled plasma.

In aqueous solution, Factor IX was shown to have a broad optimum pH for shelf stability. The stability at the key temperatures used in real-time or accelerated storage trials, such as 5±3° C., 25±2° C. and 40±2° C., is relatively similar at any pH between 5.8 to 7.6. Whilst any pH within this range can be used in the context of the present invention the recommended pH is about 6.8. Preferably, one or two displaced buffers, as described in WO2008/084237A2, are used to maintain optimum pH.

An important aspect of buffer selection in the present invention lies in controlling the metal ions, e.g., adding calcium ions and avoiding free forms of medium-strength and strong ligands. Accordingly, buffers such as displaced buffers, are preferably selected among weak ligands in relation to calcium ion binding. In this respect, benzoate and tris(hydroxymethyl)aminomethane (TRIS) are particularly preferred buffers. Thus the buffer could be benzoate or TRIS especially benzoate and TRIS. The concentration of each buffer is usually in the range between 1 to 100 mM, preferably between 5 to 50 mM, most preferably between 10 to 30 mM.

In one embodiment, an aqueous composition comprises a therapeutically relevant concentration of Factor IX, further characterized in that:
(i) the composition comprises calcium ions at concentration up to 1 mM, preferably between 0.1 to 0.7 mM, most preferably between 0.2-0.5 mM or 0.4-0.6 mM;
(ii) the composition is substantially free of the free forms of excipients which are medium-strength ligands or strong ligands;
(iii) the pH of the composition is adjusted to 5.8 to 7.6 e.g. about 6.8;
(iv) the composition is kept in a sealed non-glass container, such as a polypropylene or polyethylene container.

It was found beneficial to add to such compositions a small amount of a strong ligand, such as EDTA. It is critical, however, that the concentration of the strong ligand does not exceed the concentration of calcium ion present in the composition. Preferably the concentration of the strong ligand is less than half of the concentration of calcium ion, for example one tenth of the concentration of the calcium ion. The strong ligand is then practically absent in its free (i.e. not bound to metal ion) form. It is believed that the simultaneous presence of calcium ion and the strong ligand has the benefit of removing traces of other ions (such as cupric or ferric ions) which may otherwise be present in the composition as contaminants and contribute to detrimental oxidation or aggregation processes. Therefore, in another embodiment of the invention, an aqueous composition comprises a therapeutically relevant concentration of Factor IX, further characterized in that:
(i) the composition comprises calcium ions at concentration up to 1 mM, preferably between 0.1 to 0.7 mM, most preferably between 0.2-0.5 mM or 0.4-0.6 mM;
(ii) the composition is substantially free of the free forms of excipients which are medium-strength ligands or strong ligands;
(iii) the pH of the composition is adjusted to 5.8 to 7.6 e.g. about 6.8;
(iv) the composition comprises a strong ligand at a concentration no higher than that of calcium ions; the preferred strong ligand is EDTA;
(v) the composition is kept in a sealed non-glass container, such as a polypropylene or polyethylene container.

Preferred compositions comprise a buffer system based on a combination of benzoate ion and tromethamine (TRIS). Therefore, in another embodiment, an aqueous composition comprises a therapeutically relevant concentration of Factor IX, further characterized in that:
(i) the composition comprises calcium ions at concentration up to 1 mM, preferably between 0.1 to 0.7 mM, most preferably between 0.2-0.5 mM or 0.4-0.6 mM;
(ii) the composition comprises benzoate ion and TRIS, each at concentration between 1 to 100 mM, preferably between 5 to 50 mM, most preferably between 10 to 30 mM;
(iii) the composition is substantially free of the free form of excipients which are medium-strength ligands or strong ligands;
(iv) the pH of the composition is adjusted to 5.8 to 7.6 e.g. about 6.8;
(v) the composition is kept in a sealed non-glass container, such as a polypropylene or polyethylene container.

In yet another embodiment of the present invention, an aqueous composition comprises a therapeutically relevant concentration of Factor IX, further characterized in that:
(i) the composition comprises calcium ions at concentration up to 1 mM, preferably between 0.1 to 0.7 mM, most preferably between 0.2-0.5 mM or 0.4-0.6 mM;
(ii) the composition comprises benzoate ion and TRIS, each at concentration between 1 to 100 mM, preferably between 5 to 50 mM, most preferably between 10 to 30 mM;
(iii) the composition is substantially free of excipients which are medium-strength ligands or strong ligands;
(iv) the pH of the composition is adjusted to 5.8 to 7.6 e.g. about 6.8;
(v) the composition comprises a strong ligand at a concentration no higher than that of calcium ions; the preferred strong ligand is EDTA;
(vi) the composition is kept in a sealed non-glass container, such as a polypropylene or polyethylene container.
   Compositions in all embodiments have preferably one or more of the following features:
(i) the composition is sterile and filled aseptically into a suitable container such as a sterile vial, ampoule or pre-filled syringe; the sterility can be achieved by filtering the composition prior to the final filling to the container using an appropriate filter or membrane, such as a 0.22 μm filter or a 0.45 μm filter; the composition may also contain a pharmaceutically acceptable preservative, such as phenol, m-cresol or benzyl alcohol;
(ii) the composition comprises a pharmaceutically acceptable surfactant, such as such as polysorbate 20, polysorbate 40, polysorbate 60, polysorbate 80, poloxamer 188 or poloxamer 407;
(iii) the osmolarity of the composition is adjusted to the required level using either pharmaceutically acceptable ionic species, preferably sodium chloride, or pharmaceutically acceptable non-ionic species such as mannitol or 1,2-propanediol.

Another embodiment of the invention is an optimized aqueous composition of Factor IX which comprises a therapeutically relevant concentration of Factor IX, further characterized in that:
(i) the composition comprises calcium ions at concentration between 0.1 to 0.7 mM e.g. 0.4-0.6 mM;
(ii) the composition comprises benzoate ion and TRIS, each at concentration between 10 to 25 mM;
(iii) the pH of the composition is adjusted to 5.8 to 7.6 e.g. 6.8;
(iv) the composition comprises EDTA at a concentration substantially lower than that of the calcium ion;
(v) the composition comprises polysorbate 80 at a concentration between 10 to 50 mg/l;
(vi) the composition is sterile;
(vii) the composition is kept in a non-glass container, such as polypropylene and polyethylene container.

Benzoic acid or its salts, such as sodium or potassium salt, can be used as the source of benzoate anion. Either TRIS base or TRIS hydrochloride can be used as a source of TRIS. Calcium chloride is the preferred source of calcium ions, but other soluble salts of calcium can also be used.

In another aspect of the present invention, an aqueous composition comprises a therapeutically relevant concentration of Factor IX, further characterized in that the potency of Factor IX is maintained in such composition within 10% of the potency in the freshly prepared composition following incubation at 5±3° C. for a minimum of 20 weeks, as determined by the APTT test, whilst manifesting no significant self-activation, as determined by the observation of a response time of >200 s in the NAPTT test.

In another aspect of the present invention, an aqueous composition comprises a therapeutically relevant concentration of Factor IX, further characterized in that the potency of Factor IX is maintained in such composition within 10% of the potency in the freshly prepared composition following incubation at 25±2° C. for a minimum of 20 weeks, as determined by the APTT test, whilst manifesting no significant self-activation, as determined by the observation of a response time of >200 s in the NAPTT test.

In another aspect of the present invention, an aqueous composition comprises a therapeutically relevant concentration of Factor IX, further characterized in that the potency of Factor IX is maintained in such composition within 10% of the potency in the freshly prepared composition following incubation at 5±3° C. for a minimum of 1 year, as determined by the APTT test, whilst manifesting no significant self-activation, as determined by the observation of a response time of >200 s in the NAPTT test.

“Control Composition” is defined herein as a composition possessing the same components and excipients in the same concentrations without being subjected to the storage conditions. It is also understood that storage for substantial periods of time is expected to be subjected to varied conditions. Typical storage at 25° C. will actually include changes or variations in temperature typical for room temperature storage. Typically, where the composition is subjected to a controlled stability test, the temperature will be maintained within 3° C. of the stated temperature. However, it is understood that the testing can also be conducted with a product obtained, for example, from a commercial lot at the point of sale or at the time of administration. In these instances, it is understood that the storage temperatures, particularly room temperature storage, may not be as tightly controlled and may vary by 10° C. or more. Such variations in storage conditions are intended to be included within the scope of the claims.

Preferably the composition comprises water, a therapeutically effective amount of Factor IX, calcium ions, EDTA, and one or more displaced buffers and an alkali metal ion, and, optionally, a surfactant and/or a preservative. In preferred embodiments, the composition consists essentially of these components. A composition consisting essentially of the stated components is intended to exclude compositions that contain excipients or additives that result in the reduction of Factor IX potency under the conditions of storage. For example, a composition consisting essentially of the stated components is intended to exclude a composition which contains an excipient having a functional group with a pKa value within 1 pH unit of the pH of the formulation and/or a strong ligand in amounts which exceed the concentration of free metal ions present in the formulation.

Factor IX is preferably present in the composition in an amount between 50 and 1000 e.g. 50 and 500 IU/ml, preferably between 50 and 250 IU/ml. Alternatively the amount could be between 25 and 50 IU/ml. The concentration could be between 50 and as much as 5000 IU/ml. IU is understood to mean international units, as defined by the WHO. The invention is applicable to recombinant Factor IX, Factor IX purified from pooled plasma and molecules comprising domains with amino acid sequence identical to the native human Factor IX as well as analogues in which mutations of the amino acid sequence have been implemented without significantly affecting the therapeutic activity.

Surfactants can also be optionally added to the composition. Preferred surfactants include polysorbate 20, polysorbate 60, polysorbate 80, Poloxamer 188 or Poloxamer 407. The surfactants can preferably be added in an amount up 10 mg/ml, such as up to 5 mg/ml, such as 3 mg/ml. Preferably, the composition comprises polysorbate 80 at a concentration between 10 to 50 mg/L or Poloxamer 188 at a concentration between 0.2 to 3 mg/mL.

The composition can also optionally comprise a preservative, such as those approved for use in drug products. Preferred preservatives can be selected from the group comprising phenol, m-cresol, benzyl alcohol, propylparaben, benzalkonium chloride and benzethonium chloride.

In embodiments, a diol or polyol can be added, such as selected from 1,2-propanediol, glycerol, mannitol, sorbitol, trehalose, raffinose or sucrose and such as at a concentration of at least 100 mM. Preferably, the above composition comprises 1,2-propanediol or mannitol, most preferably 1,2-propanediol, for example at a concentration between 100 mM to 1 M, most preferably between 200 mM to 500 mM.

Such substances are capable of advantageously modifying the tonicity of the composition.

Suitably the tonicity of the composition is not modified by inclusion of NaCl. Thus for example the composition may be free or substantially free of NaCl (e.g. less than 20 mM e.g. less than 2 mM e.g. less than 0.2 mM NaCl).

The invention makes possible a stable aqueous formulation of Factor IX used by hemophiliac patients to manage their potentially life-threatening condition. These patients have to receive an intravenous infusion of the Factor IX two or three times a week and this is typically done at home by the patient, without medical supervision. Many of these patients are children and the procedure of self-administration is made more complex by the need to re-constitute the Factor IX when supplied as a freeze-dried powder with sterile water for injection. The stable aqueous product facilitated by this invention would be supplied ready to use in a pre-filled syringe, considerably simplifying this procedure of self-administration. The benefits to patient convenience are clear, and there is also potential for improved safety as the patient is no longer involved in the complex procedure of sterile reconstitution of the protein at the correct concentration and dosage.

Further aspects of the invention include a sealed glass container comprising an aqueous composition of the invention.

An aqueous composition of the invention is also claimed per se.

By “an aqueous composition of the invention” is meant an aqueous composition comprising Factor IX in a buffer and calcium ions at a concentration of less than or equal to 1 mM and wherein the composition is free or substantially free of the free forms of excipients which are medium-strength ligands or strong ligands.

### EXPERIMENTATION

### Testing Methods:

Factor IX activity measurement (APTT test) was performed as described in the EP monograph (01/2008:20711; 2.7.11. Assay of human Coagulation Factor IX). The self-activation of Factor IX measurement was performed as described in the EP monograph (01/2008:1223; 2.6.22. Activated coagulation factors). The residual potency measurements were determined relative to the 4^{th} International Standard.

### Material:

MONONINE® (Aventis Behring) freeze-dried human coagulation Factor IX (1000 IU) was used as the starting material in all experiments. Upon re-constitution in 10 ml of water (i.e. recommended reconstitution prior to use) the preparation contains 10 mM histidine, 66 mM sodium chloride and 165 mM mannitol. In therapeutic use, contents after reconstitution should be administered at room temperature within three hours after reconstitution. MONONINE® is prepared from pooled human plasma.

All new formulations were prepared at 100 IU/mL Factor IX activity. The stabilized formulations were prepared by a three-step dialysis of the reconstituted MONONINE® product against the new formulation and subsequent adjustment of volume to achieve the required specific activity of the Factor IX.

### Example 1

This Example shows the stability of the reconstituted MONONINE® following reconstitution at 100 IU/ml. The formulations were prepared in Type I borosilicate glass vials and sealed with a crimp top. A gradual loss of potency was observed in the liquid composition both at 25° C. and at 37° C. The rate of potency loss was greater at 37° C. The self activation of Factor IX (NAPTT test) was not assessed in this experiment.

| | |
|---|---|
| Time/ | Residual potency (%) |

| weeks | 25 ± 2° C. | 37 ± 2° C. |
|---|---|---|
| 0 | 100 | 100 |
| 4 | 70.8 | 17 |
| 8 | | 4 |
| 10 | 39.6 | |
| 16 | 20.8 | |

### Example 2

This Example shows the stability, measured by the APTT potency test, of the liquid formulations of Factor IX in the compositions containing:
Potassium benzoate (10 mM)
TRIS (10 mM)
1,2-Propanediol
Tween 80 (25 mg/l)
Calcium chloride (concentration between 0-2 mM, as specified in the Table below)
EDTA (concentration always at one tenth of that of calcium chloride, e.g. 0.05 mM if calcium chloride is 0.5 mM)
   All compositions were tested both in glass (type I borosilicate) and in plastic (polypropylene) containers. The NAPTT test was not carried out in this experiment, so no direct measure of the self-activation of Factor IX in the compositions was obtained. However, the self-activation is evident in a number of samples where the apparent residual potency, measured by the APTT test, increased considerably above 100%. This is due to the fact that the activated Factor IX results in a higher signal of the APTT test than the non-activated Factor IX. Factor IX is gradually activated during the APTT test to produce the signal, so any pre-activated material in the mixture will cause an increase in the signal. Two trends can therefore be observed during the storage trials: (1) decrease in potency due to loss of native structure of Factor IX and (2) apparent increase in potency due to the presence of self-activated form of the remaining native Factor IX. The key stability trends are clear from the Table below.

The results (Table below) show the combined effect of calcium ions and glass surface on the stability and the apparent rate of self activation. The rate of self activation was considerably higher in the glass container than in a polypropylene container, as evidenced by residual potency values noticeably higher than 100%, for example higher than 120%, in some cases higher than 200% or even 300%. In addition, increased level of calcium ions further contributed to the rate of self-activation. The degree of self-activation appeared to be minimal in the glass containers in the absence of calcium ions (this is consistent with observations reported in U.S. Pat. No. 5,770,700), but the stability, especially at 25° C. was relatively poor. In contrast, very limited self-activation was observed in the polypropylene vials, which allowed the use of calcium ions in the compositions to improve the stability of the native Factor IX. Some signs of self activation were observed after prolonged incubation using higher level (2 mM) of calcium even in the plastic containers.

So, the use of plastic container and either 0.5 mM or 1 mM calcium ions, together with the specifically selected background composition based on benzoate ion, TRIS, 1,2-propanediol and Tween 80 resulted in apparently stable Factor IX with a minimal loss of native structure and a minimal self-activation. Absence of calcium ions resulted in poorer stability, whereas the presence of 2 mM calcium ions apparently resulted in a slight self-activation at 25° C. It may be noted that a concentration of 2 mM calcium ions in glass apparently resulted in significant self-activation at 25° C.

| | | Residual potency (%) | | | | | | | |
|---|---|---|---|---|---|---|---|---|---|
| | | Polypropylene container | | | | Glass container | | | |
| | | 0 mM | 0.5 mM | 1 mM | 2 mM | 0 mM | 0.5 mM | 1 mM | 2 mM |
| Time | | Ca²⁺ | Ca²⁺ | Ca²⁺ | Ca²⁺ | Ca²⁺ | Ca²⁺ | Ca²⁺ | Ca²⁺ |
| 37° C. | | | | | | | | | |
| 0 | weeks | 100 | 100 | 100 | 100 | 100 | 100 | 100 | 100 |
| 4 | weeks | 27 | 73 | 47 | 79 | 33 | 90 | 79 | 81 |
| 10 | weeks | 2 | 32 | 18 | 54 | 8 | 61 | 61 | 67 |
| 16 | weeks | 0 | 8 | 10 | 21 | 1 | 39 | 50 | 48 |
| 25° C. | | | | | | | | | |
| 0 | weeks | 100 | 100 | 100 | 100 | 100 | 100 | 100 | 100 |
| 10 | weeks | 58 | 98 | 95 | 102 | 40 | 43 | 108 | 341 |
| 16 | weeks | 48 | 98 | 102 | 107 | 28 | 58 | 254 | 250 |
| 28 | weeks | 22 | 89 | 104 | 124 | 6 | 73 | 41 | 72 |
| 2-8° C. | | | | | | | | | |
| 0 | weeks | 100 | 100 | 100 | 100 | 100 | 100 | 100 | 100 |
| 10 | weeks | 95 | 101 | 101 | 100 | 102 | 98 | 93 | 109 |
| 16 | weeks | 95 | 100 | 100 | 105 | 107 | 124 | 154 | 209 |
| 28 | weeks | 97 | 97 | 102 | 111 | 76 | 212 | 274 | 374 |

### Example 3

In this experiment, the effect of the concentration of the calcium ion was studied both on the stability (APTT test) and on the self-activation (NAPTT test) of Factor IX. The background formulation was the same as that in Example 2:
Potassium benzoate (10 mM)
TRIS (10 mM)
1,2-Propanediol
Tween 80 (25 mg/l)
Calcium chloride (concentration between 0-1 mM, as specified in the Table below)
EDTA (concentration always at one tenth of that of calcium chloride, e.g. 0.05 mM if calcium chloride is 0.5 mM)

All compositions were tested in plastic (polypropylene) containers. The degree of self-activation was estimated by the NAPTT test as described in the EP monograph (01/2008:1223; 2.6.22. Activated coagulation factors). The initial (non-activated) time is typically between 200-250 s. Self-activation will decrease the NAPTT time. Shortening the time to values >150 is not considered significant. Shortening the NAPTT time to <150 indicates significant self-activation that would be unacceptable for a product release.

No drop below the 150 s limit of the NAPTT time was observed in any of the samples studied. However, a slight decrease in the NAPTT time was observed after 20 weeks at 25° C. in the presence of 1 mM Ca²⁺, indicating that 0.5 mM calcium ion is preferable to 1 mM. The potency (measured by the APTT test) was generally worse in the absence of calcium.

So, the stability of Factor IX was demonstrated to be best in the selected background formulation if the liquid composition was kept in a polypropylene container and if it contained a low (around 0.5 mM) concentration of calcium ions.

| | |
|---|---|
| Time/ | Residual potency (%)/NAPTT time (s) |

| weeks | 0 mM Ca²⁺ | 0.5 mM Ca²⁺ | 1 mM Ca²⁺ |
|---|---|---|---|
| 37° C. | | | |

| | | | | |
|---|---|---|---|---|
| 0 | weeks | 100%/>200 s | 100%/>200 s | 100%/>200 s |
| 2 | weeks | 39%/>200 s | 86%/>200 s | 89%/>200 s |
| 6 | weeks | 17%/>200 s | 59%/>200 s | 63%/>200 s |
| 14 | weeks | 2%/>200 s | 17%/>200 s | Not done |
| 20 | weeks | 0%/>200 s | 10%/>200 s | 17%/>200 s |

25° C.

| | | | | |
|---|---|---|---|---|
| 0 | weeks | 100%/>200 s | 100%/>200 s | 100%/>200 s |
| 6 | weeks | 103%/>200 s | 103%/>200 s | 106%/>200 s |
| 14 | weeks | 76%/>200 s | 95%/>200 s | 103%/>200 s |
| 20 | weeks | 63%/>200 s | 96%/>200 s | 100%/155 s |

2-8° C.

| | | | | |
|---|---|---|---|---|
| 0 | weeks | 100%/>200 s | 100%/>200 s | 100%/>200 s |
| 6 | weeks | 97%/>200 s | 107%/>200 s | 106%/>200 s |
| 14 | weeks | 87%/>200 s | 97%/>200 s | 102%/>200 s |
| 20 | weeks | 117%/>200 s | 101%/>200 s | 98%/>200 s |

While this invention has been particularly shown and described with references to preferred embodiments thereof, it will be understood by those skilled in the art that various changes in form and details may be made therein without departing from the scope of the invention encompassed by the appended claims.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents and patent applications mentioned throughout the specification of the present invention are herein incorporated in their entirety by reference.

The invention embraces all combinations of preferred and more preferred groups and suitable and more suitable groups and embodiments of groups recited above.

1. An aqueous therapeutic composition sealed in a non-glass container comprising Factor IX in a buffer and calcium ions at a concentration of between about 0.1 to 1 mM and wherein the composition is free or substantially free of the free forms of excipients which are medium-strength ligands or strong ligands.

2. A composition of claim 1 wherein the non-glass container is made from one or more materials selected from polypropylene, polyethylene, a polypropylene-polyethylene copolymer, polycarbonate, polystyrene or thermoplastic polyester.

3. (canceled)

4. A composition of claim 1 wherein the calcium ions are present in an amount between 0.2 to 0.7 mM.

5. A composition of claim 1 wherein the pH is between 5.8 and 7.6.

6. A composition of claim 1 comprising tris(hydroxymethyl)aminomethane (TRIS) or benzoate as buffer.

7. A composition of claim 1 further comprising a small amount of a strong ligand in an amount that does not exceed the concentration of calcium ion present in the composition.

8. A composition of claim 1 wherein the buffer comprises a buffer system based on a combination of benzoate ion and TRIS, each at a concentration between 1 to 100 mM.

9. An aqueous composition comprising a therapeutically relevant concentration of Factor IX, further characterized in that:
(i) the composition comprises calcium ions at concentration of between about 0.1 mM to 1 mM;
(ii) the composition comprises benzoate ion and TRIS, each at concentration between 1 to 100 mM;
(iii) the composition is substantially free of the free form of excipients which are medium-strength ligands or strong ligands;
(iv) the pH of the composition is adjusted to between 5.8 and 7.6;
(v) the composition comprises a strong ligand at a concentration no higher than that of calcium ions in the composition;
(vi) the composition is stored in a sealed non-glass container.

10. The composition of claim 1 further characterized by one or more of the following features:
(i) the composition is sterile and filled aseptically into a suitable container such as a sterile vial, ampoule or pre-filled syringe; said sterility optionally achieved by filtering the composition prior to the final filling to the container using an appropriate filter or membrane;
(ii) the composition further comprises a pharmaceutically acceptable preservative;
(iii) the composition further comprises a pharmaceutically acceptable surfactant optionally at a concentration between 10 to 50 mg/ml;
(iv) the osmolarity of the composition is adjusted using either pharmaceutically acceptable ionic species, or pharmaceutically acceptable non-ionic species.

11. A method of stabilizing Factor IX in an aqueous solution comprising storing an aqueous composition according to claim 1 in a non-glass container for at least 7 days.

12. A method of claim 11 wherein the sealed non-glass container is made from one or more materials selected from polypropylene, polyethylene, a polypropylene-polyethylene copolymer, polycarbonate, polystyrene and thermoplastic polyester.

13. A composition according to claim 1 which is storage-stable at 2-8° C. for a minimum of 26 weeks comprising a therapeutically effective amount of Factor IX and an aqueous medium having a Factor IX potency after 26 weeks of storage at 5° C. of at least 90% of the Factor IX potency of a Control Composition.

14. The composition of claim 13, wherein the Factor IX potency after 26 weeks of storage at 2-8° C. is at least 95% of the Factor IX potency of a Control Composition.

15. The composition of claim 13, wherein the Factor IX potency after 52 weeks of storage at 2-8° C. is at least 90% of the Factor IX potency of a Control Composition.

16. The composition of claim 13, wherein the Factor IX potency after 52 weeks of storage at 2-8° C. is at least 95% of the Factor IX potency of a Control Composition.

17. The composition of claim 13, wherein the Factor IX potency after 52 weeks of storage at 2-8° C. is at least 98% of the Factor IX potency of a Control Composition.

18. A composition of claim 1 characterized by a response time of >200 seconds in a non-activated partial prothrombin time (NAPTT) test.

19. An aqueous composition comprising Factor IX a buffer and calcium ions at a concentration of between about 0.1 mM to 1 mM and wherein the composition is free or substantially free of the free forms of excipients which are medium-strength ligands or strong ligands.

20. A composition of claim 3 wherein the calcium ions are present in an amount between 0.4 to 0.6 mM.

21. A composition of claim 6 comprising TRIS and benzoate as buffer.

22. A composition of claim 1 wherein the pH is about 6.8.

23. A composition of claim 7 wherein the strong ligand is EDTA.

24. A composition of claim 7 wherein the strong ligand is less than half of the concentration of calcium ion in the composition.

25. A composition of claim 7 wherein the strong ligand is about one tenth of the concentration of the calcium ion in the composition.
